(19) United States
(12) Patent Application Publication
(10) Pub. No.: US 2005/0015138 A1
(43) Pub. Date: Jan. 20, 2005

(54) Stent made of a material with low radio-opaqueness

(76) Inventors: Andreas Schuessler, Pfinztal-Berghausen (DE); Martin Stobel, Karlsruhe (DE); Ralf Steiner, Pforzheim (DE); Erik Flaxmeier, Karlsbad (DE)

Correspondence Address: MCDONALD HOPKINS CO., LPA, 2100 BANK ONE CENTER, 600 SUPERIOR AVENUE, E., CLEVELAND, OH, 44114-2653, US

(21) Appl. No.: 10/850,892

(22) Filed: May 21, 2004

(30) Foreign Application Priority Data
May 22, 2003 (DE) 103 23 210.9-43

(51) Int. Cl. A61F 2/06

(52) U.S. Cl. 623/1.16; 623/1.34

(57) ABSTRACT

A stent to be implanted in a living body with an essentially tube-shaped wall (12) made of material with low radio-opaqueness, which is designed through the shaping of openings (14) as flexible wall structure. In order to provide a stent (10) and a process for its manufacturing, which can be better recognized in case of research with x-radiation, a material (22) with high radio-opaqueness is inserted inside the flexible wall structure (20).

The invention concerns a stent to be implanted in a living body with an essentially tube-shaped wall made from a material of low radio-opaqueness and a weak radio-opaque material, which is constructed as a flexible wall structure through the shaping of holes. Furthermore, the invention concerns a process for the manufacturing of such a stent. This application claims priority to German patent application serial number 103 23 210.9 filed on May 22, 2003.

Stents are used in order to protect a lumen and channel of a living body, such as blood vessels, the oesophagus, the scapha and kidney channels, by means of expansion of the essentially tube-shaped wall structure of the stent in the interior of the channel against collapsing and closing. In addition, stents are used as carriers of medicine, which enable at least a local therapy in channel of the body. What's more, stents can be adopted as aneurism stent and endoprosthesis for intracelebral vascular aneurism or as intraluminal stent.

Stents of this type show a multitude of partition walls which are interconnected by means of cell connectors and nodal points. The partition walls are made of a flexible material, such as nitinol or stainless steel so that the entire stent features a flexible wall structure that can adjust itself to the curving and diameter of a lumen.

In the application of such stents, the problem arises that materials such as nitinol or stainless steel show an insufficient visibility when conducting x-ray research and, that is why, the position of the stents are very hard to determine in research of this nature.

In order to solve this problem, stents are available that feature an area of a larger surface or special markers of radio-opaque material at the endpoints of their wall structure. The markers are either fastened by means of a riveting procedure or welded onto the endpoints of the stents in the capacity of paddles.

The invention is based on the task of supplying a stent and a process for its manufacturing, which is easier to recognize in the case of research with x-radiation. Particularly, the exact position of the stent and its shape within the channel of the body should be recognizable.

The task is, according to the invention, is solved by means of a stent of the initially named type, where the material of higher radio-opaqueness and a strong radio-opaque material inside and at the flexible wall structure has been mounted. Furthermore, the task is solved by a manufacturing process of a stent with the step: affixing, providing and supplying a material of high radio-opaqueness within the flexible wall structure of the stent.

The flexible wall structure of a stent is subtly dissected and its partition walls, as well as cell connectors feature only a small surface. Consequently, the wall structure as such appears inappropriate to mount a material of high radio-opaqueness onto it. However, the invention is based on the perception that the overall area of a flexible wall structure of a stent is by all means sufficiently large, and, subsequently, the material that is mounted, provided and distributed at least range-wise and at least over a section of the surface of the wall structure, characterized with comparatively high radio-opaqueness in case of x-ray research, is sufficiently recognized.

It is particularly beneficial in the solution according to the invention that the essentially entire stent can be seen in x-ray research as such and not only its endpoints. For that reason, the exact position and moulding inside a channel of the body can be recognized in the case of a stent according to the invention.

Furthermore, an insufficiently or unequally widened wall structure can be detected in x-ray research in the case of a used stent, according to the invention, since the essentially entire flexible wall structure can be seen and, as a result, local compacting in comparison to local expansions of this flexible wall structure can become visible by means of the material of high radio-opaqueness mounted in the wall structure.

For this reason, the flexible wall structure is shaped with partition walls and/or cell connectors and the material of high radio-opaqueness is at least on one of the partition walls or cell connectors mounted in the case of a beneficial further education of the invention.

It is in addition beneficial when the partition wall and cell connectors, onto which the material of high radio-opaqueness is mounted, is shaped with a correspondingly increased base. The increased base facilitates the mounting of the material of high radio-opaqueness and offers, furthermore, itself a shielding against x-radiation.

The material of high radio-opaqueness is in addition beneficially set up inside or at the flexible wall structure in at least in an open area or antum. The material can, in this way, be embedded in the wall structure so that the flexible attitude of the wall structure does not change due to the inserted material. Hence, a new construction of the wall structure is not necessary, but on the other hand one can revert to known and proven structures.

The open area, according to a preferred embodiment of the invention, is beneficially punctiform or linear-shaped. The points or lines can be formed at the cell connectors and/or at the partition walls. The cell connectors and partition walls become in this way individually visible resulting in the fact that particularly clear conclusions can be drawn from the moulding and design of an inserted stent.

Furthermore, at least an open area is beneficially shaped as cavity or as passage opening. The material of high radio-opaqueness can be inserted particularly steadily through such a cavity or passage opening.

At least an open area should be formed by moulding of the raw material of the essentially cylindrical or tube-shaped wall and, subsequently, should be inserted into the open area in such a way that, following the succeeding shaping of the openings inside the flexible wall structure, sections of the material of high radio-opaqueness remain inside the flexible wall structure. The manufacturing cost for mounting the material of high radio-opaqueness can, in this way, be kept comparatively low.

In this procedure, the open area should at least area-wise be shaped as channel in circumference direction, axially and/or spirally on the raw material of the essentially tube-shaped wall. The material of high radio-opaqueness is equally distributed over the entire wall of a stent that is manufactured in such a manner, and, at the same time, the required manufacturing cost is comparatively speaking low.

The open area can be formed particularly cost-efficiently by means of laser cutting, laser ablation techniques, mechanical grinding, milling and/or eroding.

The material with high radio-opaqueness can be beneficially mounted on at least an open area by means of laser welding. In addition, it is beneficial when the surface of the material with high radio-opaqueness (in essence) succinctly secludes the tube-shaped wall structure through the surface. The external shape of such a stent corresponds with the well-known stents so that no further problems can emerge when inserting and shaping the stent in a channel of the body.

The material with high radio-opaqueness can be structured as bead-molding or flat ribbon in a particularly simple manner. The diameter, widths and densities of approx. 10 μm to approx. 200 μm can be determined as particularly beneficial dimensions for the points and lines of the material with high radio-opaqueness mounted according to the invention.

The flexible wall structure of the stent is preferably formed from nitinol or a nitinol alloy according to the invention.

The material with high radio-opaqueness preferably comprises tantalum, niobium, gold, platinum, wolfram or an alloy thereof.

In the following section, embodiments of a stent according to the invention are clarified on the basis of the enclosed schematic drawings. It shows:

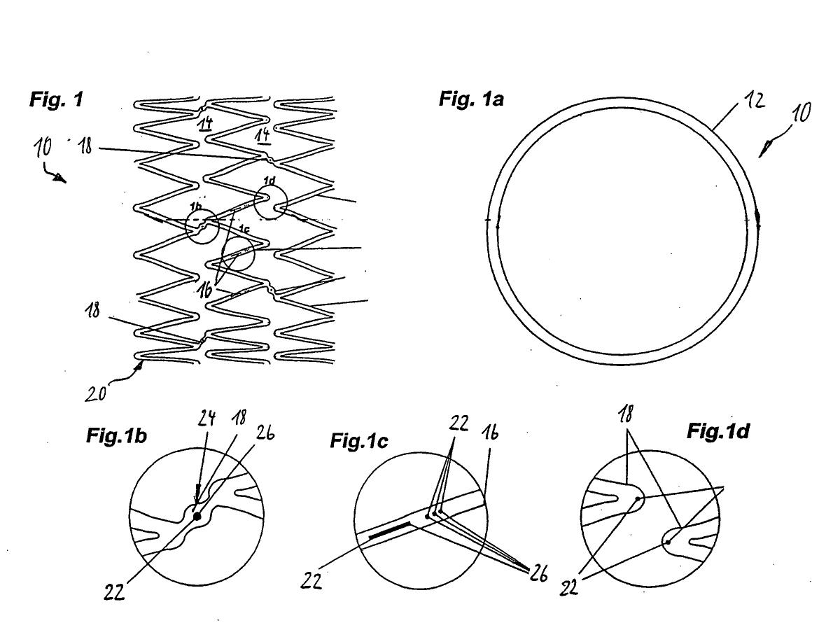

FIG. 1 a lateral view of a first embodiment of a stent according to the invention,

FIG. 1a a side view on the stent according to FIG. 1,

FIG. 1 b a detailed view of the section 1b, according to FIG. 1,

FIG. 1c a detailed view of the section 1c, according to FIG. 1,

FIG. 1d a detailed view of the section 1d, according to FIG. 1,

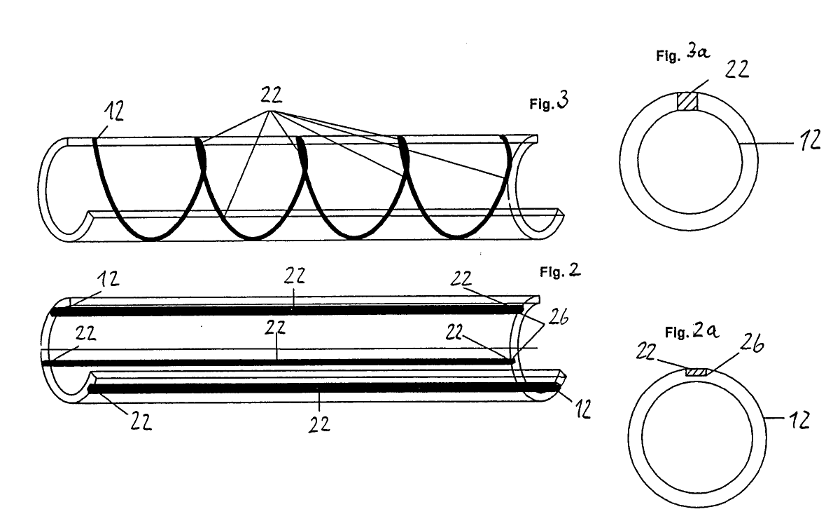

FIG. 2 a partially broken up lateral view of a second embodiment of a stent, according to the invention,

FIG. 2a the side view on the stent, according to FIG. 2,

FIG. 3 a partially broken up lateral view of a third embodiment of a stent, according to the invention and

FIG. 3a the side view on the stent, according to FIG. 3.

A first embodiment of a stent 10 is illustrated in FIGS. 1 and 1a to 1d, which is shaped for the implantation into a living body and is formed with an essentially tube-shaped, hollow cylinder-shaped wall 12. In other words, wall 12 is given a shape in the form ready for use, which corresponds with the form of the vessel or lumen in which the stent 10 will be applied. The wall 12 in itself can be shaped in a tube and/or made out of a sheet plate, flat wire and/or wire. Partition walls 16 and cell connectors 18, which make up together a flexible wall structure 20 inside the wall 12, are shaped in the wall by means of openings or sections 14.

The partition walls 16 and the cell connectors 18 are preferably made from nitinol, a material that shows only a low radio-opaqueness (that is, a comparatively high permeability for x-rays). In order to make the stent 10 and particularly its entire wall structure 20 better visible during x-ray research, a material 22 with high radio-opaqueness is mounted on the partition walls 16 and the cell connectors 18 inside the flexible wall structure 20.

In order to insert the material 22 with high radio-opaqueness, areas 24 with increased base have been formed on the partition walls 16 and the cell connectors 18, respectively, in which drillings and grooves were shaped as open areas 26 in the wall structure 20, according to the embodiment of FIG. 1 and 1a to 1d. These individual areas 24 are preferably distributed over the essentially entire surface of the stent 10.

The open areas 26 of the stent 10 of the first embodiment have been formed as passage openings when creating the openings 14 by means of a laser welding process. Subsequently, the material 22 with high radio-opaqueness was affixed in the open areas 26 by means of a laser welding process. It is preferable that this laser welding process of a tube takes place, which is for example interfused by a fluid (e.g. a cooled liquidity), so that a laser beam going through the tube wall is refracted on the inside of the tube and/or that the tube is cooled by means of the fluid that is flowing through. Furthermore, the stent 10 can be made of flat wire or wire, respectively, whereby a netting is preferably formed.

An embodiment of the stent 10 is illustrated in the FIGS. 2 and 2a, in which grooves in a lateral direction towards wall 12 have been shaped through a mechanical grinding procedure in the tube material of the wall 12. Subsequently, a material with high radio-opaqueness (in other words, in relationship with the basic material of the stent, for example, nitinol, of lower permeability for x-rays, that preferably shows a permeability [dB] which is equal to about half or less of the permeability of the basic material) is welded in or embedded or affixed, respectively in the open areas 26.

In a connecting manufacturing process that is not illustrated, the openings 14 are cut out or shaped, respectively, in the wall 12. In this manner, individual sections of the material 22 with high radio-opaqueness can be shaped at least partially in the remaining partition walls 16 and cell connectors 18.

An embodiment of a stent 10 is illustrated in FIGS. 3 and 3a, where the open areas 26 have been shaped in the form of a spiral as passageway in the tube material of wall 12. A material 22 with high radio-opaqueness has been applied in the form of a flat ribbon in these open areas 26 and has also been affixed by means of laser welding. Based on connective tube material made of material with low and high radio-opaqueness displayed in FIG. 3, the openings 14 are subsequently cut out and the partition walls 16 and 18 remain, which on their part show individual sections with the material 22 with high radio-opaqueness.

In all displayed embodiments, the surface of the material 22 with high radio-opaqueness is flush or near flush with the surface of the essentially tube-shaped wall structure 20.

According to the present invention, the stents 10 can be manufactured from stainless steel or cobalt-chrome tantalum alloy. In this way, the stents are preferably widened through a widening installation such as the balloon catheter. It is preferable that the invention or a preferred embodiment thereof is used in the case of balloon-expanded stents made of stainless steel, tantalum, niobium or cobalt alloys. It is possible that stents made of other materials, such as polymers, self-degradable materials (e.g. lactic acid or derivatives), as well as stents made of nitinol (nickel-titanium alloys) and/or of other self-expandable materials and (preferably temperature-dependent) shape-memory materials, are used.

1. A stent for the implantation in a living body comprising: an essentially tube-shaped wall made of a first material with lower radio-opaqueness, said wall having openings to provide a flexible wall structure to the stent, and a second material with high radio-opaqueness affixed to the flexible wall structure.

2. The stent according to claim 1, wherein the flexible wall structure further comprises at least one partition wall and at least one cell connector and wherein the second material with high radio-opaqueness is affixed to at least one selected from the group consisting of: the partition walls or the cell connectors.

3. The stent according to claim 2, wherein the second material with high radio-opaqueness is affixed to both the partition wall and cell connector, said partition wall and cell connector having a correspondingly increased base relative to the flexible wall structure.

4. The stent according to claim 1, wherein the flexible wall structure further comprises at least one open area and wherein the second material with high radio-opaqueness is affixed to the flexible wall structure at least one of the open areas.

5. The stent according to claim 4, wherein the open area is formed in shape selected from the group consisting of: a point, a line, a cavity and a passageway.

6. The stent according to claim 2, wherein the flexible wall structure further comprises at least one open area and wherein the second material with high radio-opaqueness is affixed to the flexible wall structure at least one of the open areas.

7. The stent according to claim 4, wherein the open area is molded in the essentially tube-shaped wall and, subsequently, the second material with high radio-opaqueness is affixed on the molded open area in such a manner portions of the second material with high radio-opaqueness are integral to the flexible wall structure.

8. The stent according to claim 7, wherein the open area is formed axially, spirally or circumferentially along the essentially tube-shaped wall.

9. The stent according to claim 4, wherein the open area has been constructed by means of laser welding, laser ablation techniques, mechanical grinding, milling and/or eroding.

10. The stent according to claim 4, wherein the second material with high radio-opaqueness is affixed to at least one open area by means of laser welding.

11. The stent according to claim 1, wherein an exterior surface of the second material with high radio-opaqueness is substantially flush with an exterior surface of the essentially tube-shaped wall.

12. The stent according to claim 1, wherein the second material with high radio-opaqueness is shaped as bead-molding or flat ribbon, said bead molding or flat ribbon having a diameter or width between 10 μm to 200 μm.

13. The stent according claim 1, wherein the flexible wall structure is formed from nitinol or a nitinol alloy.

14. The stent according to claim 1, wherein the second material with high radio-opaqueness is tantalum, niobium, gold, platinum, wolfram or an alloy thereof.

15. A process for manufacturing a stent to be implanted in a living body comprising the following steps: providing an essentially tube-shaped wall made of a first material with low radio-opaqueness; shaping openings in the essentially tube shaped wall to create a flexible wall structure; and affixing a second material with high radio-opaqueness on the flexible wall structure.

16. The process according to claim 15, wherein the shaping of the flexible wall structure also includes creation of at least one partition wall and cell connector and wherein the second material with high radio-opaqueness is affixed to at least one of the partition walls or cell connectors.

17. The process according to claim 16, wherein the shaping of the flexible wall structure also includes creation of an increased base at the partition wall and the cell connector and wherein the second material with high radio-opaqueness is affixed to the increased base.

18. The process according claim 15, wherein the second material with high radio-opaqueness is affixed to at least one open area on the flexible wall structure.

19. The process according to claim 18, wherein the open area is provided formed as a pointy shape, a lined shape, a cavity or a passageway.

20. The process according claim 16, wherein the second material with high radio-opaqueness is affixed to at least one open area on the flexible wall structure.

21. The process according claim 18, wherein the open area is formed in the essentially tube-shaped wall and wherein the second material with high radio-opaqueness is inserted into the open area in such a manner that a portion of the second material with high radio-opaqueness will remain inside the flexible wall structure even after the step of shaping openings to create the flexible wall structure.

22. The process according to claim 21, wherein the open area is formed as a circumferential, axial or spiral groove in the essentially tube-shaped wall.

23. The process according claim 18, wherein the open area is formed by means of laser welding, laser ablation techniques, mechanical grinding, milling and/or eroding.

24. The process according claim 18, wherein the second material with high radio-opaqueness is affixed to the open area by means of laser welding.

25. The process according claim 15, wherein an exterior surface of the second material with high radio-opaqueness is affixed in such a manner that it is flush with an exterior surface of the flexible wall structure.

26. The process according claim 15, wherein the second material with high radio-opaqueness is affixed as bead-molding or flat ribbon, said bead-molding or flat ribbon having a diameter or width between 10 μm and 200 μm.

27. The process according to claim 15, wherein the flexible wall structure is made of nitinol or a nitinol alloy.

28. The process according claims 15, wherein the second material with high radio-opaqueness is selected from the group consisting of: tantalum, niobium, gold, wolfram or an alloy or mixture thereof.
